(19) United States
(12) Patent Application Publication
(10) Pub. No.: US 2024/0207617 A1
(43) Pub. Date: Jun. 27, 2024

(54) SYSTEMS AND METHODS FOR INDIVIDUALIZED TARGETING OF DEEP BRAIN STIMULATION

(71) Applicants: Washington University, St. Louis, MO (US); The Regents of the University of Michigan, Ann Arbor, MI (US); Emory University, Atlanta, GA (US)

(72) Inventors: Peter Brunner, St. Louis, MO (US); Markus Adamek, St. Louis, MO (US); Jon Willie, St. Louis, MO (US); Eric Leuthardt, St. Louis, MO (US); Enrico Opri, Ann Arbor, MI (US); Svjetlana Miocinovic, Atlanta, GA (US)

(73) Assignees: Washington University, St. Louis, MO (US); The Regents of the University of Michigan, Ann Arbor, MI (US); Emory University, Atlanta, GA (US)

(21) Appl. No.: 18/526,386

(22) Filed: Dec. 1, 2023

Related U.S. Application Data
(60) Provisional application No. 63/385,701, filed on Dec. 1, 2022.

(51) Int. Cl. A61N 1/36

(52) U.S. Cl. CPC A61N 1/36139; A61N 1/36067; A61N 1/36157; A61N 1/36171

(57) ABSTRACT

Among the various aspects of the present disclosure is the provision for systems and methods for individualized targeting of deep brain stimulation (DBS). In addition, methods for monitoring the treatment of a subject using a DBS system are described.

### CROSS-REFERENCE TO RELATED APPLICATIONS

This application claims priority from U.S. Provisional Application Ser. No. 63/385,701 filed on Dec. 1, 2022, which is incorporated herein by reference in its entirety.

### STATEMENT REGARDING FEDERALLY SPONSORED RESEARCH OR DEVELOPMENT

This invention was made with government support under EB018783, NS109103, EB026439, NS123103, NS097576, and MH120194 awarded by the National Institutes of Health. The government has certain rights in the invention.

### MATERIAL INCORPORATED-BY-REFERENCE

Not applicable.

### FIELD OF THE INVENTION

The present disclosure generally relates to systems and methods for electrical stimulation for individual targeting in deep brain stimulation.

### BACKGROUND OF THE INVENTION

Approximately 3 million adults in the US have active epilepsy, with a third being insufficiently controlled by medication alone. These patients require alternative treatment options. One such alternative treatment option is the continuous electrical deep-brain-stimulation (DBS) of the centromedian nucleus of the thalamus (CMN). CMN-DBS has recently emerged as a promising therapeutic intervention for patients with drug-resistant epilepsy causing tonic-clonic seizures, secondarily generalized seizures, or Lennox-Gastaut syndrome. However, parameter selection for seizure control remains empiric, poorly understood, and time-consuming, with no established consensus on the optimal stimulation location. For example, early investigations suggested that stimulating the ventrolaterally located parvocellular part of CMN improves clinical outcomes. However, a recent study favors the anteromedial CMN and neighboring mediodorsal and parafascicular nuclei. These discrepancies in the literature highlight a clear need for neurophysiological biomarkers of therapy, as changes in seizure control through postoperative programming take weeks to months to observe.

### SUMMARY OF THE INVENTION

Among the various aspects of the present disclosure is the provision for systems and methods for electrical stimulation for individualized targeting in deep brain stimulation.

Briefly, therefore, the present disclosure is directed to systems and methods that facilitate the administration of individually targeted electrical stimulation in deep brain stimulation.

In one aspect, an EEG system for facilitating the placement of a deep brain stimulation (DBS) device within the brain of a subject to treat a brain disorder is disclosed. The DBS device is configured for implantation within a brain region of the subject and includes at least two stimulation electrodes configured to deliver a stimulation to the brain region of the subject. The EEG system includes an EEG sensing device configured for placement over a scalp of the subject. The EEG sensing device includes a plurality of EEG electrodes and the EEG sensing device is configured to detect a plurality of EEG signals at the scalp of the subject. The EEG system further includes a computing device operatively coupled to the DBS device and the EEG sensing device. The computing device includes at least one processor configured to operate the DBS device to deliver the stimulation to the brain region of the subject; receive the plurality of EEG signals from the EEG sensing device before and after the stimulation; transform the plurality of EEG signals received before and after the stimulation into an EEG response biomarker, the EEG response biomarker indicative of a potential extent of neural network interaction accessible by the DBS device; and display the EEG response marker to an operator of the EEG system. In some aspects, the EEG response biomarker includes a root-mean-square value or a Z-score of an EEG response to the stimulation. In some aspects, EEG response to the stimulation is evaluated within a 500 μs window after the delivery of the stimulation by the DBS device. In some aspects, the stimulation includes a series of single stimulation pulses delivered at a pulse frequency ranging from about 1 Hz to about 190 Hz. In some aspects, the single stimulation pulses include pulses delivered at 6 mA intensity, 200 μs pulse duration, and 120 seconds stimulation duration. In some aspects, the pulse frequency is selected from about 1 Hz, about 25 Hz, and about 50 Hz. In some aspects, the EEG sensing device includes a wearable scalp EEG electrode array. In some aspects, the system is configured to operate intraoperatively during implantation of the at least two stimulation electrodes of the DBS device, postoperatively after implantation of the at least two stimulation electrodes of the DBS device; and any combination thereof. In some aspects, the at least one processor is further configured to operate the DBS device to deliver at least two different stimulations to the brain region of the subject, each stimulation delivered by a different pair-wise combination of the at least two electrodes; compare at least two EEG biomarkers, each EEG biomarker corresponding to each pair-wise combination of the at least two electrodes; select a final pair-wise combination of stimulation electrodes for subsequent DBS treatment of the subject, the final pair-wise combination of stimulation electrodes corresponding to the maximum EEG biomarker value; and display to the operator of the system the final pair-wise combination of stimulation electrodes for use in subsequent DBS treatments.

In another aspect, a computer-implemented method of positioning and configuring the operation of a DBS device within the brain region of a subject is disclosed. The method includes positioning an EEG sensing device that includes a plurality of EEG electrodes and configured to detect a plurality of EEG signals on a scalp of the subject, and implanting the DBS device comprising at least two stimulation electrodes within the brain region of the subject. The method further includes obtaining at least two candidate EEG biomarker values during the implantation of the DBS device within the brain region of the subject. The at least two candidate EEG biomarker values are obtained for at least one DBS treatment parameter. The method further includes comparing, using the computing device, the at least two EEG biomarker values to identify a maximum EEG biomarker value and configuring the operation of the DBS device to match the at least one treatment parameter of the DBS device associated with the maximum EEG biomarker. In some aspects, obtaining at least two candidate EEG biomarker values includes, for each candidate EEG biomarker value, operating the DBS device to deliver the stimulation to the brain region of the subject, receiving the plurality of EEG signals from the EEG sensing device before and after the stimulation, transforming the plurality of EEG signals received before and after the stimulation into one candidate EEG response biomarker, and displaying the one candidate EEG response marker to an operator of the EEG system. The one candidate EEG response biomarker is indicative of a potential extent of neural network interaction accessible by the DBS device. In some aspects, at least one DBS treatment parameter is selected from one or more positions of the at least one stimulation electrodes within the brain region of the subject, one or more pair-wise combinations of the at least two stimulation electrodes of the DBS device, one or more combinations of DBS operating parameters, and any combination thereof. In some aspects, the DBS operating parameters are selected from a pulse frequency, a pulse duration, a pulse amplitude, a treatment duration, and any combination thereof.

In another aspect, a computer-implemented method of monitoring an efficacy of a DBS treatment of a brain disorder of a subject using an EEG system that includes a computing device operatively coupled to an EEG sensing device configured to detect a plurality of EEG signals on a scalp of the subject is disclosed. The method includes administering a first DBS treatment and obtaining a first EEG biomarker using the EEG system, administering a second DBS treatment and obtaining a second EEG biomarker using the EEG system, obtaining a change in the second EEG biomarker value relative to the first EEG biomarker value, and evaluating the efficacy of the DBS treatment based on a predetermined correlation of the efficacy and the change in the second EEG biomarker value relative to the first EEG biomarker value. In some aspects, the brain region includes a centromedian thalamic nucleus region. In some aspects, the brain disorder includes one of Parkinson's disease and epilepsy.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5A is an illustration of lead placement within the centromedian nucleus of the thalamus. R0 represents the most distal lead.

FIG. 5B is a set of plots of the spatial distribution of the root-mean-square (RMS) of the evoked scalp response. Involvement of the most distal lead (R0) causes increased EEG activity in the parietal cortex compared to stimulation of the more superior leads (e.g. stimulation of the R2-R3 pair).

FIG. 5C is a graph of the stimulation response in electrode Pz. Stimulation of the most distal contact (R0) causes increased responses 150 ms after stimulation, compared to other stimulation locations. The shaded gray background shows the area used for the calculation of RMS. Shaded areas for induced responses represent the standard error of the mean.

FIG. 5D is a graph of the mean RMS for location Pz across stimulation conditions. Stimulation conditions including the most distal contact (R0) cause significantly higher responses compared to other stimulation locations (** p<0.01, Wilcoxon rank-sum test, Bonf. corr., N=120 per condition, pairwise comparison of all combinations). Bars represent the standard error of the mean.

FIG. 6A is a schematic of the Standardized Workflow for the Advanced Selection And Programming (ASAP) protocol showing that an intraoperative scalp EEG is used to record patient-specific network engagement caused by electric stimulation of the implanted DBS leads.

FIG. 6B is a schematic of the Standardized Workflow for the Advanced Selection And Programming (ASAP) protocol showing that short, single pulses of electrical current identify the amount of network engagement measured through the scalp EEG.

FIG. 6C is a schematic of the Standardized Workflow for the Advanced Selection And Programming (ASAP) protocol showing how the optimal location for therapeutic stimulation is determined.

FIG. 6D is a schematic of the Standardized Workflow for the Advanced Selection And Programming (ASAP) protocol showing that the location with the largest response will be used for initial therapeutic stimulation.

FIG. 7A is a plot of the spatial distribution of the root-mean-square (RMS) of the evoked scalp responses in Patient 1. Stimulation of the centromedian nucleus evokes increased activity in the parietal lobe.

FIG. 7B is a set of graphs as an example of evoked responses on the scalp electrode Pz across stimulated CMN leads in Patient 1. The grey shaded area is used to calculate the RMS values shown in the topographic plot in FIG. 7A. The shaded area corresponds to the standard error of the mean (N=120). Right to the evoked responses, the RMS value for channel Pz across all 120 trials is presented on the topography plots in FIG. 7A. Bar represents standard error.

FIG. 8A is a plot of the spatial distribution of the root-mean-square (RMS) of the evoked scalp responses in Patient 2. Stimulation of the centromedian nucleus evokes increased activity in the parietal lobe.

FIG. 8B is a set of graphs as an example of evoked responses on the scalp electrode Pz across stimulated CMN leads in Patient 2. The grey shaded area is used to calculate the RMS values shown in the topographic plot in FIG. 8A. The shaded area corresponds to the standard error of the mean (N=120). Right to the evoked responses, the RMS value for channel Pz across all 120 trials is presented on the topography plots in FIG. 8A. Bar represents standard error.

FIG. 9A is a plot of the spatial distribution of the root-mean-square (RMS) of the evoked scalp responses in Patient 3. Stimulation of the centromedian nucleus evokes increased activity in the parietal lobe.

FIG. 9B is a set of graphs as an example of evoked responses on the scalp electrode Pz across stimulated CMN leads in Patient 3. The grey shaded area is used to calculate the RMS values shown in the topographic plot in FIG. 9A. The shaded area corresponds to the standard error of the mean (N=120). Right to the evoked responses, the RMS value for channel Pz across all 120 trials is presented on the topography plots in FIG. 9A. Bar represents standard error.

FIG. 10A is a schematic illustration showing the color coding of the evoked responses along the implanted lead in various graphs herein.

FIG. 10B contains a series of schematic illustrations showing reconstructions of the implanted lead locations on a probabilistic nucleus segmentation atlas using FreeSurfer. Leads selected for therapeutic intervention are shown in red. Numbers represent patients in the experimental cohort.

FIG. 11 is a schematic illustration of the quantification of changes in scalp responses to single stimuli to single-pulse stimulation administered at different scalp locations.

FIG. 12A is a graph summarizing the differences in scalp responses to single stimuli between scalp locations FP1 and F7 (see FIG. 11); traces are colored to represent measurements obtained using different electrode pairs as illustrated in FIG. 10A.

FIG. 12B is a graph summarizing the differences in scalp responses to single stimuli between scalp locations F8 and T4 (see FIG. 11); traces are colored to represent measurements obtained using different electrode pairs as illustrated in FIG. 10A.

FIG. 12C is a graph summarizing the differences in scalp responses to single stimuli between scalp locations P3 and O1 (see FIG. 11); traces are colored to represent measurements obtained using different electrode pairs as illustrated in FIG. 10A.

FIG. 12D is a graph summarizing the differences in scalp responses to single stimuli between scalp locations T3 and T4 (see FIG. 11); traces are colored to represent measurements obtained using different electrode pairs as illustrated in FIG. 10A.

FIG. 12E is a graph summarizing the differences in scalp responses to single stimuli between scalp locations F7 and T3 (see FIG. 11); traces are colored to represent measurements obtained using different electrode pairs as illustrated in FIG. 10A.

FIG. 12F is a graph summarizing the differences in scalp responses to single stimuli between scalp locations T4 and T6 (see FIG. 11); traces are colored to represent measurements obtained using different electrode pairs as illustrated in FIG. 10A.

FIG. 12G is a graph summarizing the differences in scalp responses to single stimuli between scalp locations FP2 and F4 (see FIG. 11); traces are colored to represent measurements obtained using different electrode pairs as illustrated in FIG. 10A.

FIG. 12H is a graph summarizing the differences in scalp responses to single stimuli between scalp locations T3 and T5 (see FIG. 11); traces are colored to represent measurements obtained using different electrode pairs as illustrated in FIG. 10A.

FIG. 12I is a graph summarizing the differences in scalp responses to single stimuli between scalp locations T6 and O2 (see FIG. 11); traces are colored to represent measurements obtained using different electrode pairs as illustrated in FIG. 10A.

FIG. 12J is a graph summarizing the differences in scalp responses to single stimuli between scalp locations F4 and P4 (see FIG. 11); traces are colored to represent measurements obtained using different electrode pairs as illustrated in FIG. 10A.

FIG. 12K is a graph summarizing the differences in scalp responses to single stimuli between scalp locations T5 and O1 (see FIG. 11); traces are colored to represent measurements obtained using different electrode pairs as illustrated in FIG. 10A.

FIG. 12L is a graph summarizing the differences in scalp responses to single stimuli between scalp locations FP1 and F3 (see FIG. 11); traces are colored to represent measurements obtained using different electrode pairs as illustrated in FIG. 10A.

FIG. 12M is a graph summarizing the differences in scalp responses to single stimuli between scalp locations P4 and O2 (see FIG. 11); traces are colored to represent measurements obtained using different electrode pairs as illustrated in FIG. 10A.

FIG. 12N is a graph summarizing the differences in scalp responses to single stimuli between scalp locations FP2 and F8 (see FIG. 11); traces are colored to represent measurements obtained using different electrode pairs as illustrated in FIG. 10A.

FIG. 12O is a graph summarizing the differences in scalp responses to single stimuli between scalp locations F3 and P3 (see FIG. 11); traces are colored to represent measurements obtained using different electrode pairs as illustrated in FIG. 10A.

FIG. 12P is a graph summarizing the differences in scalp responses to single stimuli between scalp locations FZ and PZ (see FIG. 11); traces are colored to represent measurements obtained using different electrode pairs as illustrated in FIG. 10A.

FIG. 13 is a schematic illustration of the quantification of changes in scalp responses to 50 Hz stimulation administered at different scalp locations.

FIG. 14 is a schematic illustration showing the process of assessing changes in scalp responses to 50 Hz stimulations at different scalp locations.

FIG. 15A is a graph summarizing the differences in scalp responses to 50 Hz stimulations between scalp locations FP1 and F7 (see FIG. 11); traces are colored to represent measurements obtained using different electrode pairs as illustrated in FIG. 10A.

FIG. 15B is a graph summarizing the differences in scalp responses to 50 Hz stimulations between scalp locations F8 and T4 (see FIG. 11); traces are colored to represent measurements obtained using different electrode pairs as illustrated in FIG. 10A.

FIG. 15C is a graph summarizing the differences in scalp responses to 50 Hz stimulations between scalp locations P3 and O1 (see FIG. 11); traces are colored to represent measurements obtained using different electrode pairs as illustrated in FIG. 10A.

FIG. 15D is a graph summarizing the differences in scalp responses to 50 Hz stimulations between scalp locations T3 and T4 (see FIG. 11); traces are colored to represent measurements obtained using different electrode pairs as illustrated in FIG. 10A.

FIG. 15E is a graph summarizing the differences in scalp responses to 50 Hz stimulations between scalp locations F7 and T3 (see FIG. 11); traces are colored to represent measurements obtained using different electrode pairs as illustrated in FIG. 10A.

FIG. 15F is a graph summarizing the differences in scalp responses to 50 Hz stimulations between scalp locations T4 and T6 (see FIG. 11); traces are colored to represent measurements obtained using different electrode pairs as illustrated in FIG. 10A.

FIG. 15G is a graph summarizing the differences in scalp responses to 50 Hz stimulations between scalp locations FP2 and F4 (see FIG. 11); traces are colored to represent measurements obtained using different electrode pairs as illustrated in FIG. 10A.

FIG. 15H is a graph summarizing the differences in scalp responses to 50 Hz stimulations between scalp locations T5 and O1 (see FIG. 11); traces are colored to represent measurements obtained using different electrode pairs as illustrated in FIG. 10A.

FIG. 15I is a graph summarizing the differences in scalp responses to 50 Hz stimulations between scalp locations FP1 and F3 (see FIG. 11); traces are colored to represent measurements obtained using different electrode pairs as illustrated in FIG. 10A.

FIG. 15J is a graph summarizing the differences in scalp responses to 50 Hz stimulations between scalp locations P4 and O2 (see FIG. 11); traces are colored to represent measurements obtained using different electrode pairs as illustrated in FIG. 10A.

FIG. 15K is a graph summarizing the differences in scalp responses to 50 Hz stimulations between scalp locations FP2 and F8 (see FIG. 11); traces are colored to represent measurements obtained using different electrode pairs as illustrated in FIG. 10A.

FIG. 15L is a graph summarizing the differences in scalp responses to 50 Hz stimulations between scalp locations F3 and P3 (see FIG. 11); traces are colored to represent measurements obtained using different electrode pairs as illustrated in FIG. 10A.

FIG. 15M is a graph summarizing the differences in scalp responses to 50 Hz stimulations between scalp locations FZ and PZ (see FIG. 11); traces are colored to represent measurements obtained using different electrode pairs as illustrated in FIG. 10A.

FIG. 16 a set of graphs of signals from various scalp locations before and after single-pulse stimulations.

FIG. 17A is a graph summarizing the differences in scalp responses to 50 Hz stimulations between scalp locations FP1 and F7 (see FIG. 11); traces are colored to represent measurements obtained using different electrode pairs as illustrated in FIG. 10A.

FIG. 17B is a graph summarizing the differences in scalp responses to 50 Hz stimulations between scalp locations F8 and T4 (see FIG. 11); traces are colored to represent measurements obtained using different electrode pairs as illustrated in FIG. 10A.

FIG. 17C is a graph summarizing the differences in scalp responses to 50 Hz stimulations between scalp locations P3 and O1 (see FIG. 11); traces are colored to represent measurements obtained using different electrode pairs as illustrated in FIG. 10A.

FIG. 17D is a graph summarizing the differences in scalp responses to 50 Hz stimulations between scalp locations T3 and T4 (see FIG. 11); traces are colored to represent measurements obtained using different electrode pairs as illustrated in FIG. 10A.

FIG. 17E is a graph summarizing the differences in scalp responses to 50 Hz stimulations between scalp locations F7 and T3 (see FIG. 11); traces are colored to represent measurements obtained using different electrode pairs as illustrated in FIG. 10A.

FIG. 17F is a graph summarizing the differences in scalp responses to 50 Hz stimulations between scalp locations T4 and T6 (see FIG. 11); traces are colored to represent measurements obtained using different electrode pairs as illustrated in FIG. 10A.

FIG. 17G is a graph summarizing the differences in scalp responses to 50 Hz stimulations between scalp locations FP2 and F4 (see FIG. 11); traces are colored to represent measurements obtained using different electrode pairs as illustrated in FIG. 10A.

FIG. 17H is a graph summarizing the differences in scalp responses to 50 Hz stimulations between scalp locations T3 and T5 (see FIG. 11); traces are colored to represent measurements obtained using different electrode pairs as illustrated in FIG. 10A.

FIG. 17I is a graph summarizing the differences in scalp responses to 50 Hz stimulations between scalp locations T6 and O2 (see FIG. 11); traces are colored to represent measurements obtained using different electrode pairs as illustrated in FIG. 10A.

FIG. 17J is a graph summarizing the differences in scalp responses to 50 Hz stimulations between scalp locations F4 and P4 (see FIG. 11); traces are colored to represent measurements obtained using different electrode pairs as illustrated in FIG. 10A.

FIG. 17K is a graph summarizing the differences in scalp responses to 50 Hz stimulations between scalp locations T5 and O1 (see FIG. 11); traces are colored to represent measurements obtained using different electrode pairs as illustrated in FIG. 10A.

FIG. 17L is a graph summarizing the differences in scalp responses to 50 Hz stimulations between scalp locations FP1 and F3 (see FIG. 11); traces are colored to represent measurements obtained using different electrode pairs as illustrated in FIG. 10A.

FIG. 17M is a graph summarizing the differences in scalp responses to 50 Hz stimulations between scalp locations P4 and O2 (see FIG. 11); traces are colored to represent measurements obtained using different electrode pairs as illustrated in FIG. 10A.

FIG. 17N is a graph summarizing the differences in scalp responses to 50 Hz stimulations between scalp locations FP2 and F8 (see FIG. 11); traces are colored to represent measurements obtained using different electrode pairs as illustrated in FIG. 10A.

FIG. 17O is a graph summarizing the differences in scalp responses to 50 Hz stimulations between scalp locations F3 and P3 (see FIG. 11); traces are colored to represent measurements obtained using different electrode pairs as illustrated in FIG. 10A.

FIG. 17P is a graph summarizing the differences in scalp responses to 50 Hz stimulations between scalp locations FZ and PZ (see FIG. 11); traces are colored to represent measurements obtained using different electrode pairs as illustrated in FIG. 10A.

FIG. 18A contains graphs comparing ERP (left) and PSD (right) in the left hemisphere for differences between scalp responses at locations T6 and O2 (see FIG. 11); traces are colored to represent measurements obtained using different electrode pairs as illustrated in FIG. 10A.

FIG. 18B contains graphs comparing ERP (left) and PSD (right) in the left hemisphere for differences between scalp responses at locations FP1 and F3 (see FIG. 11); traces are colored to represent measurements obtained using different electrode pairs as illustrated in FIG. 10A.

FIG. 19A contains graphs comparing ERP (left) and PSD (right) in the right hemisphere for differences between scalp responses at locations T6 and O2 (see FIG. 11); traces are colored to represent measurements obtained using different electrode pairs as illustrated in FIG. 10A.

FIG. 19B contains graphs comparing ERP (left) and PSD (right) in the right hemisphere for differences between scalp responses at locations FP1 and F3 (see FIG. 11); traces are colored to represent measurements obtained using different electrode pairs as illustrated in FIG. 10A.

FIG. 20 is a graph comparing response measurements at different stimulation frequencies attributable to artifact, entrainment, and disruption of the thalamo-cortical loop.

### DETAILED DESCRIPTION OF THE INVENTION

The present disclosure is based, at least in part, on the discovery that an early (3-month) nine-fold reduction in seizure occurrence was observed for the first patient tested with the DBS system of the current disclosure. As shown herein, systems and methods are described related to an electrical DBS strategy to individually target the brain of a subject to treat brain disease.

One aspect of the present disclosure provides for an electrical DBS system to stimulate the brain of a subject.

In the present disclosure, deep brain structures are stimulated electrically using single electrical pulses or distinct frequencies while simultaneously recording electroencephalographic (EEG) activity, either during implantation or post-operatively. The responses to electrical stimulation allow mapping of effective connectivity, which can be monitored non-invasively, reducing patient risk compared to other invasive approaches that have been published. Therefore, this biomarker can be used to optimize patient treatment using quantitative measures such as Inter-Trial coherence, size of the evoked response, and amount of entrainment between the stimulated site and the recording site. Furthermore, a specialized version of a stereotactic frame is developed in which the mounting points also serve as EEG recording locations, removing the necessity for additional equipment in the operating room, saving time and risk of complications.

The present teachings include descriptions of a deep brain stimulation (DBS) system. In some aspects, the DBS system includes a DBS device, a scalp electroencephalogram (EEG) device, and a computing device. In some aspects, the DBS device can provide short electrical pulses. In some embodiments, the DBS device can provide short electrical pulses with 6 mA intensity. In some embodiments, the DBS device provides short electrical pulses with a 200 μs duration. In some embodiments, the DBS device provides short electrical pulses with a 1 Hz frequency. In another aspect, the computing system can control the DBS device. In another aspect, the computing system can transform data collected from the EEG device. In yet another aspect, the computing device of the system can transform the data collected from the EEG device into a parameter associated with neural connectivity.

The present teachings include methods for configuring a DBS device. In some aspects, configuring the DBS device includes implanting the DBS device. In another aspect, short electrical stimulation through at least one DBS electrode can be applied. In another aspect, an EEG can be simultaneously recorded during the electrical stimulation. In yet another aspect, EEG measurements can be transformed into a parameter associated with effective neural connectivity. In another aspect, the operation of the DBS device can be configured based on the parameter associated with neural connectivity. In some embodiments, the short electrical stimulation can have a 6 mA intensity. In some embodiments, the short electrical stimulation can have a 200 μs pulse duration. In some embodiments, the short electrical stimulation can have a 1 Hz pulse frequency. In some embodiments, the parameter associated with neural connectivity can be measured through the root-mean-square value of the scalp response within a 500 ms window after stimulation.

The present teachings also include methods for monitoring a brain treatment in a subject. In some aspects, the method includes implanting a DBS device. In some aspects, the method includes applying short electrical stimulation through at least one DBS electrode. In some aspects, an EEG can be simultaneously recorded during the short electrical stimulation. In some aspects, EEG measurements can be transformed into a parameter associated with effective neural connectivity. In some aspects, the operation of the DBS device can be configured based on the parameter associated with neural connectivity. In some aspects, the parameters associated with neural connectivity can be compared over time during the brain treatment. In some aspects, the operation of the DBS system can be modified based on the comparison of the parameters during the brain treatment. In some aspects, the brain treatment is DBS. In some embodiments, the short electrical stimulation can have a 6 mA intensity. In some embodiments, the short electrical stimulation can have a 200 μs pulse duration. In some embodiments, the short electrical stimulation can have a 1 Hz pulse frequency. In some embodiments, the parameter associated with neural connectivity can be measured through the root-mean-square value of the scalp response within a 500 ms window after stimulation. In some embodiments, the subject has epilepsy.

### Computing Systems and Devices

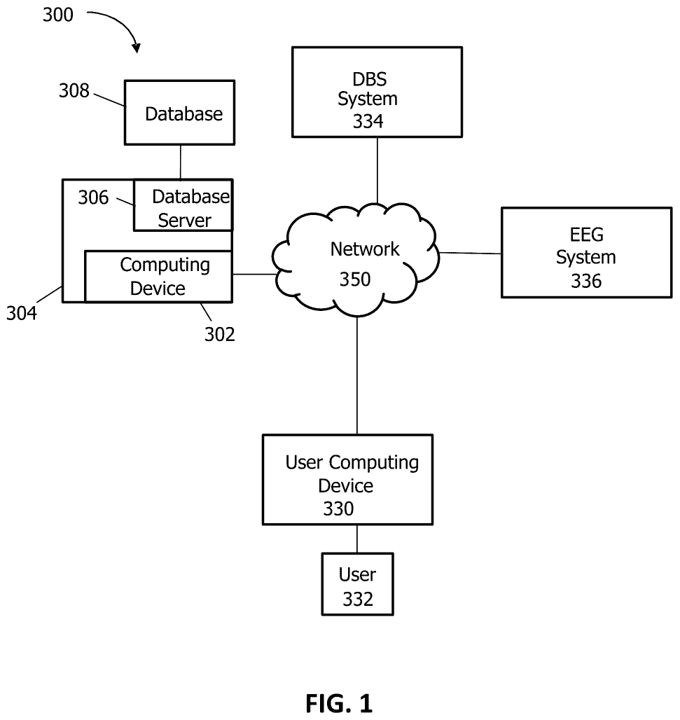

In various aspects, the disclosed DBS, EEG, and neural connectivity methods may be implemented using a computing system or computing device. FIG. 1 depicts a simplified block diagram of the system for implementing the computer-aided method described herein. As illustrated in FIG. 1, the computing device 300 may be configured to implement at least a portion of the tasks associated with the disclosed methods described herein. The computer system 300 may include a computing device 302. In one aspect, the computing device 302 is part of a server system 304, which also includes a database server 306. The computing device 302 is in communication with a database 308 through the database server 306. The computing device 302 is communicably coupled to a user computing device 330, a DBS system 334, and an EEG system 336 through a network 350. The network 350 may be any network that allows local area or wide area communication between the devices. For example, the network 350 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 330 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

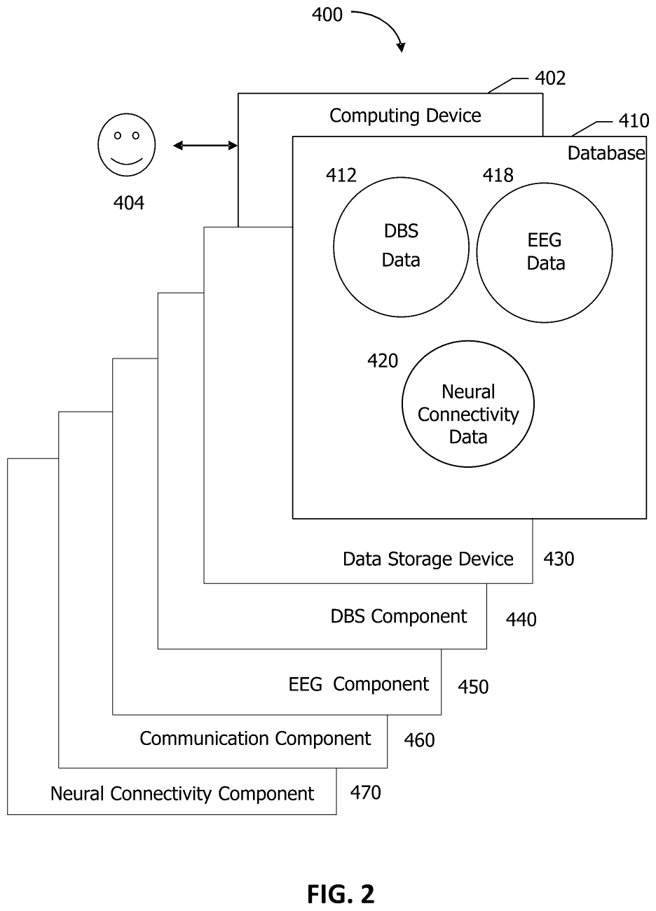

In other aspects, the computing device 302 is configured to perform a plurality of tasks associated with the disclosed computer-aided methods of performing DBS and associated EEG and neural connectivity mapping. In some aspects, the computing device 302, the user computing device 330, and/or DBS system 334 may be operatively connected via a network 350. FIG. 2 depicts a component configuration 400 of computing device 402, which includes database 410 along with other related computing components. In some aspects, computing device 402 is similar to computing device 302 (shown in FIG. 1). A user 404 may access components of computing device 402. In some aspects, database 410 is similar to database 308 (shown in FIG. 1).

In one aspect, database 410 includes DBS data 412, EEG data 418, and neural connectivity data 420. DBS data 412 may include data used to operate a DBS system as disclosed herein. Non-limiting examples of DBS data 412 include various input signals, any parameters used to control the operation of a DBS device, and any parameters defining equations or other algorithms used to implement the neural connectivity mapping as disclosed herein. EEG data 418 may include data used to perform the recording of EEG activity as disclosed herein. Non-limiting examples of EEG data 418 include measurements of background noise and/or EEG signals, any parameters defining equations and other algorithms used to implement the transformation of background noise and EEG signals into neural connectivity as disclosed herein and/or any parameters defining equations and other algorithms used to implement neural connectivity method described herein.

Computing device 402 also includes a number of components that perform specific tasks. In the exemplary aspect, computing device 402 includes a data storage device 430, a DBS component 440, an EEG component 450, a communication component 460, and a neural connectivity component 470. The DBS component 440 is configured to implement the deep brain stimulation method as described herein. The EEG component 450 is configured to implement the recording of EEG activity methods as disclosed herein. The neural connectivity component 470 is configured to implement the method of mapping the effective connectivity as described herein and to calculate EEG response biomarkers, such as the RMS or Z-score of the EEG response as described herein. The data storage device 430 is configured to store data received or generated by computing device 402, such as any of the data stored in database 410 or any outputs of processes implemented by any component of computing device 402.

The communication component 460 is configured to enable communications between computing device 402 and other devices (e.g. user computing device 330 shown in FIG. 1) over a network, such as a network 350 (shown in FIG. 1), or a plurality of network connections using predefined network protocols such as TCP/IP (Transmission Control Protocol/Internet Protocol).

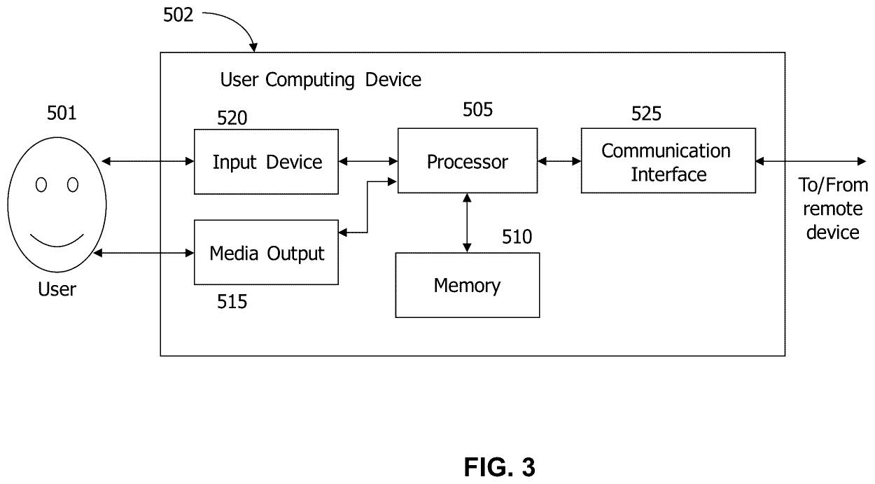

FIG. 3 depicts a configuration of a remote or user computing device 502, such as user computing device 330 (shown in FIG. 1). Computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode (OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser and client application. Web browsers enable users 501 to display and interact with media and other information typically embedded on a web page or a website from a web server. A client application allows users 501 to interact with a server application associated with, for example, a vendor or business.

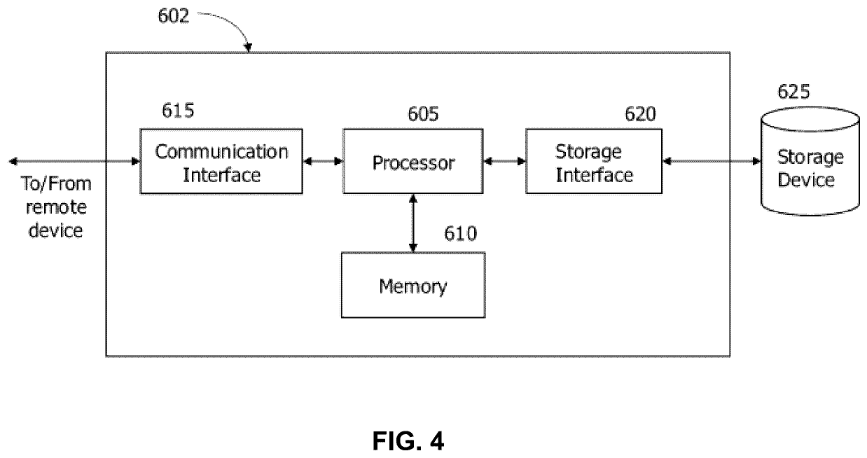

FIG. 4 illustrates an example configuration of a server system 602. Server system 602 may include, but is not limited to, database server 306 and computing device 302 (both shown in FIG. 1). In some aspects, server system 602 is similar to server system 304 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 330 (shown in FIG. 1) or another server system 602. For example, communication interface 615 may receive requests from a user computing device 330 via a network 350 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network attached storage (NAS) system.

In some aspects, processor 605 may be operatively coupled to storage device 625 via a storage interface 620. Storage interface 620 may be any component capable of providing processor 605 with access to storage device 625. Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIGS. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only and are thus not limiting as to the types of memory usable for the storage of a computer program.

The computer systems and computer-aided methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local or remote processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

The methods and algorithms of the disclosure may be enclosed in a controller or processor. Furthermore, methods and algorithms of the present disclosure can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to images or frames of a video, object characteristics, and object categorizations. Data inputs may further include sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a region within a medical image (segmentation), categorization of a type of motion, a diagnosis based on the motion of an object, motion analysis of an object, and trained model parameters ML outputs may further include: speech recognition, image or video recognition, medical diagnoses, statistical or financial models, autonomous vehicle decision-making models, robotics behavior modeling, fraud detection analysis, user recommendations and personalization, game Al, skill acquisition, targeted marketing, big data visualization, weather forecasting, and/or information extracted about a computer device, a user, a home, a vehicle, or a party of a transaction. In some aspects, data inputs may include certain ML outputs.

In some aspects, at least one of a plurality of ML methods and algorithms may be applied, which may include but are not limited to: genetic algorithms, linear or logistic regressions, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, adversarial learning, and reinforcement learning.

### Therapeutic Methods

Also provided is a process of treating, preventing, or reversing a brain disease, including but not limited to active epilepsy, in a subject in need of administration of a therapeutically effective amount of electrical brain stimulation, so as to treat the brain disease, including but not limited to treating active epilepsy or Parkinson's disease.

Methods described herein are generally performed on a subject in need thereof. A subject in need of the therapeutic methods described herein can be a subject having, diagnosed with, suspected of having, or at risk for developing a brain disease, including but not limited to epilepsy. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of electrical brain stimulation is, for example, an amount that would cause the desired therapeutic effect in a subject while minimizing undesired side effects. In various embodiments, an effective amount of an electrical brain stimulation described herein can substantially inhibit epilepsy, slow the progress of epilepsy, or limit the development of epilepsy

It will be appreciated by those skilled in the art that the unit content of treatment contained in an individual dose of each dosage form need not in itself constitute a therapeutically effective amount, as the necessary therapeutically effective amount could be reached by administration of a number of individual treatments.

Again, each of the states, diseases, disorders, and conditions, described herein, as well as others, can benefit from the methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of electrical brain stimulation can occur as a single event or over a time course of treatment. For example, electrical brain stimulation can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a brain disorder.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Centromedian Thalamic Deep Brain Stimulation for Generalized Seizures: Seeking Biomarkers to Predict Treatment Success

### Methods

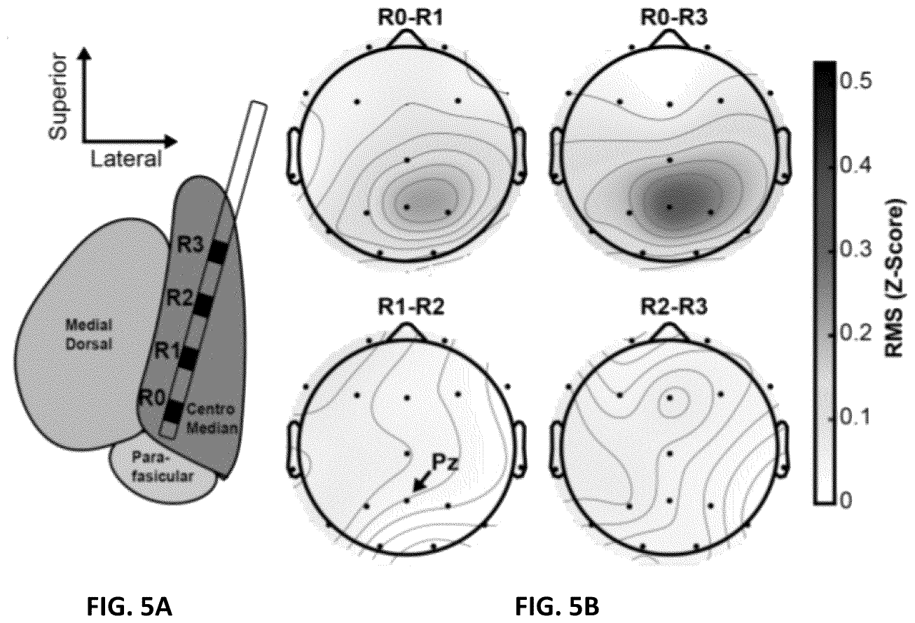

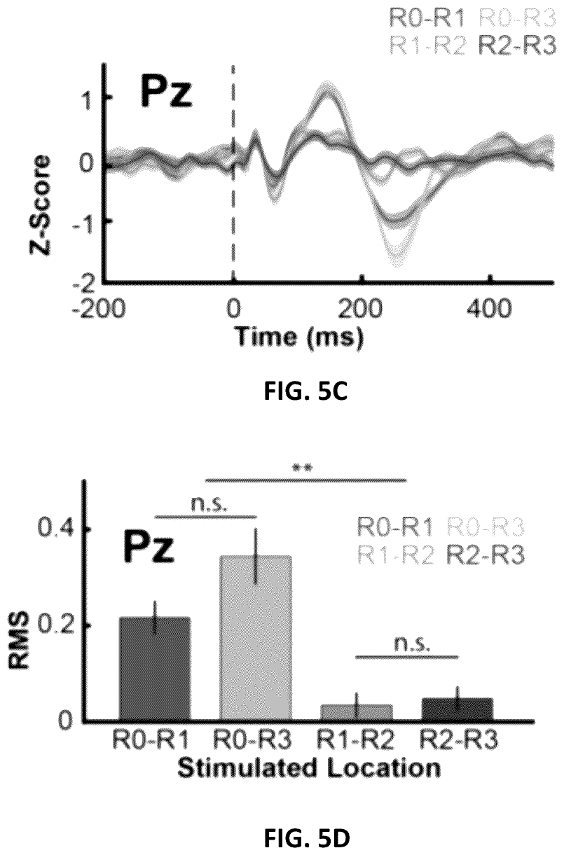

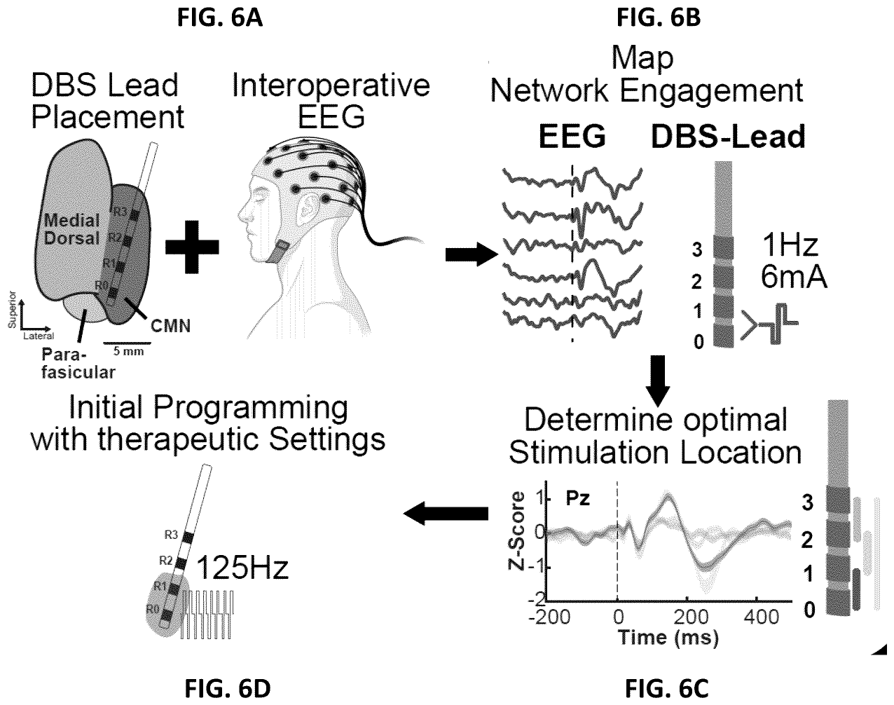

To demonstrate a method of determining the patient-specific stimulation location by observing the brain's response to single electrical stimulation pulses, the following experiments were conducted. Specifically, the potential extent of network interaction accessible through the implanted DBS leads across the CMN was quantified via intraoperatively evoked electroencephalographic (EEG) responses. For this purpose, each pairwise combination of the implanted electrode was used to administer electrical brain stimulation using short electrical pulses (6 mA intensity, 200 us pulse duration, 1 Hz pulse frequency, 120 s) while the evoked responses from the scalp-recorded EEG were simultaneously recorded. The magnitude of the induced responses for each stimulated location was quantified to infer which stimulation location caused maximum network engagement, measured through the root-mean-square value of the scalp response within a 500 ms window after stimulation (FIGS. 5A, 5B, 5C, 5D, 6A, 6B, 6C, and 6D). Finally, whether this biomarker translated into improved clinical outcomes was tested.

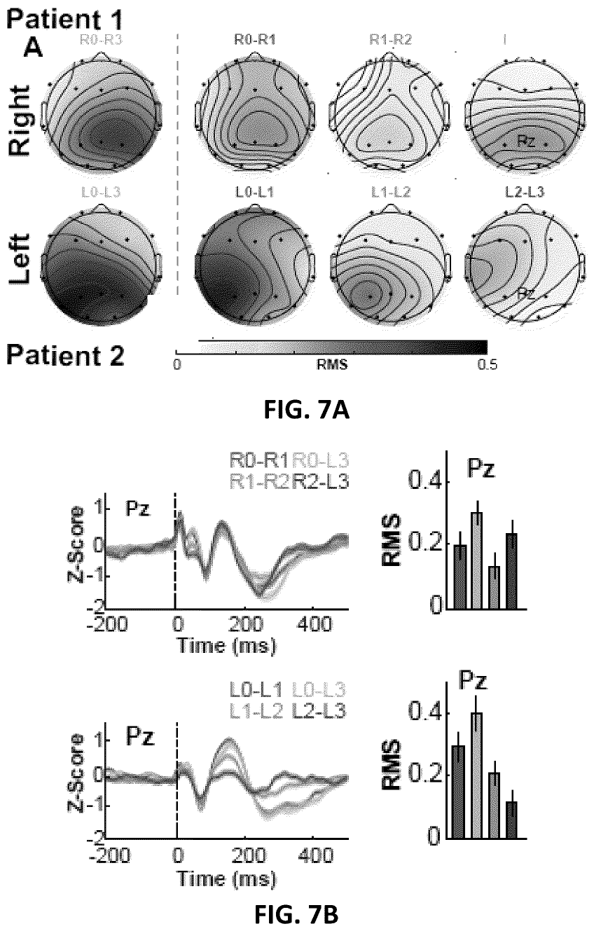

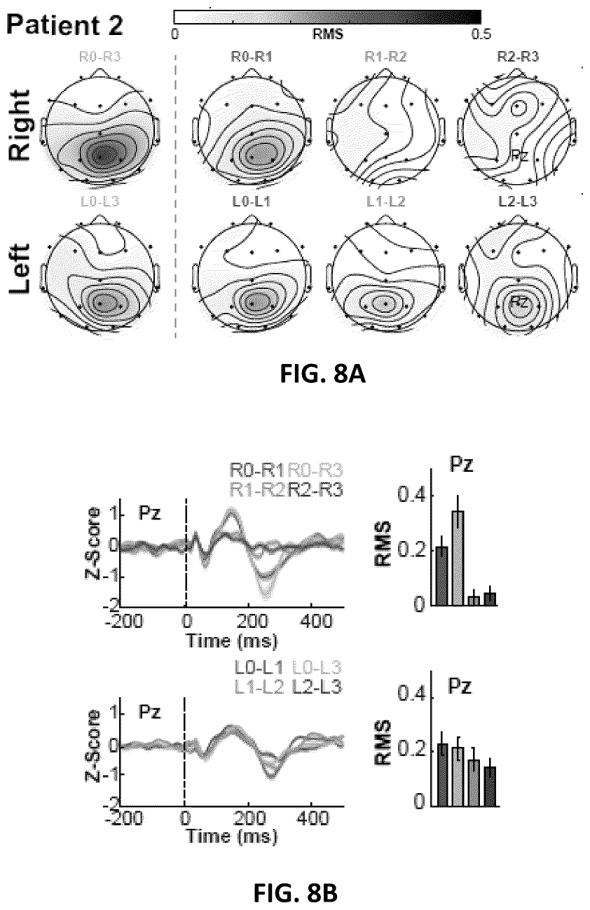

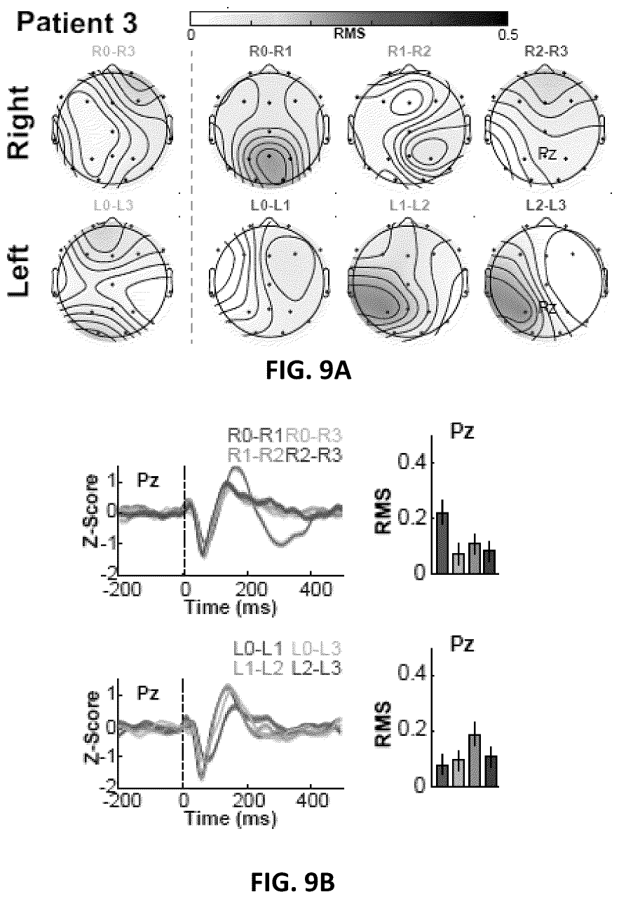

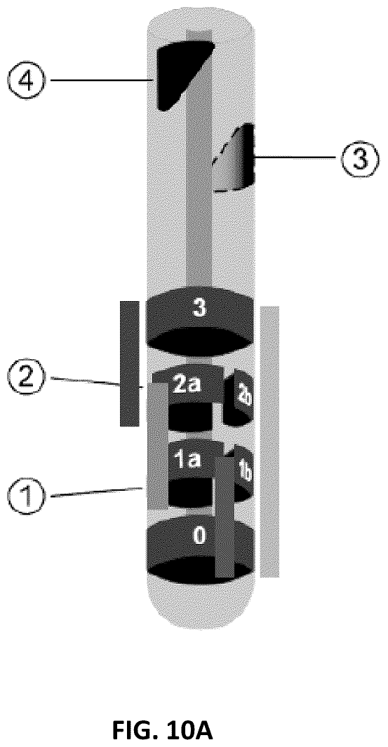

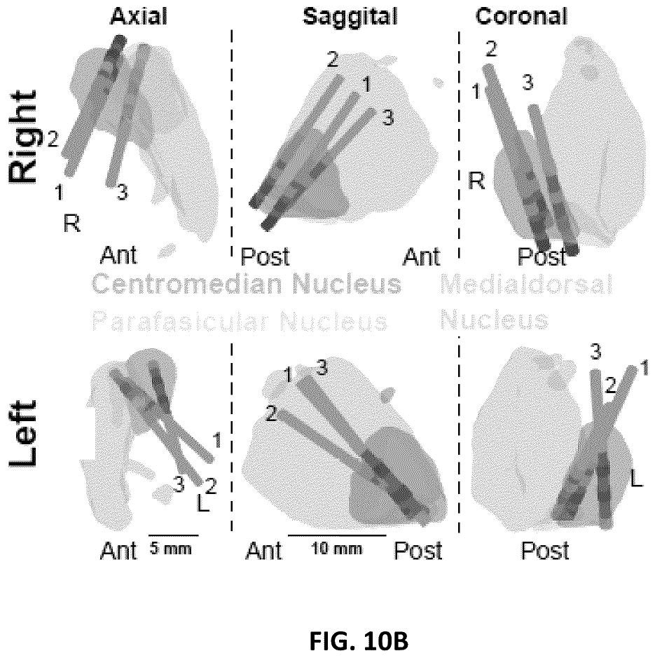

The approach described above was tested in three patients who underwent bilateral DBS of the CMN (FIGS. 7A, 7B, 8A, 8B, 9A, 9B, 10A, 10B, AND 11).

Three patients were enrolled with the ASAP protocol. Eligibility for CMN-DBS surgery was determined as part of standard clinical care. Patients were diagnosed with Generalized tonic-clonic seizures (Patient 1) or Lennox-Gastaut Syndrome (Patients 2 and 3). All patients are currently on three or more anti-epileptogenic drugs, with little changes pre- and post-implantation. All patients are also implanted with an active VNS stimulator. The initial stimulation location for each patient was determined through the ASAP protocol. Patients 1 and 2 have shown a significant reduction in seizure frequency. GTC, Generalized Tonic-Clonic; LGS, Lennox-Gastaut syndrome. KCN2A, Potassium channel mutation.

It was found that the magnitude and morphology of evoked EEG responses across lobes and hemispheres depended on the stimulation location within the CMN, highlighting the differential effect on large-scale network engagement. These results provided the basis for DBS programming to maximize the impact upon desired networks. The outcome of this initial study showed an encouraging early (3-month) nine-fold reduction in seizure occurrence for the first patient. Results from the first three patients indicate clear differences in network engagement caused by the stimulation location. The initial results suggest that some stimulation locations drive broader network engagement. Stimulation location within the centromedian nucleus of the thalamus causes differential scalp responses. Stimulation-induced evoked responses may provide a rapid biomarker to provide optimal, personalized treatment positioning for the subsequent treatment of generalized epilepsy

With further verification, the systematic characterization of CMN-DBS evoked EEG biomarkers may enable more optimal DBS programming and improved patient outcomes. The proposed approach will provide a quantitative framework upon which electrode location and parameter selection may be based.

### Example 2—Potential Biomarkers for Treatment Optimization in Patients Receiving Centromedian Thalamic Nucleus Deep Brain Stimulation

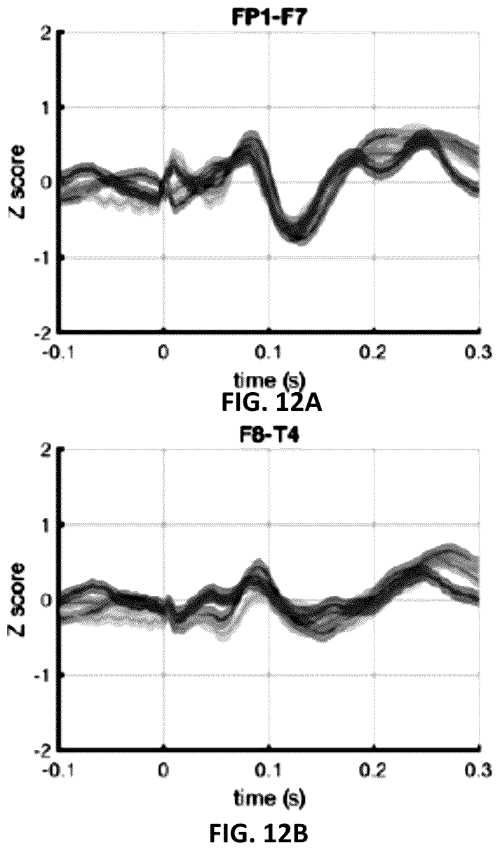

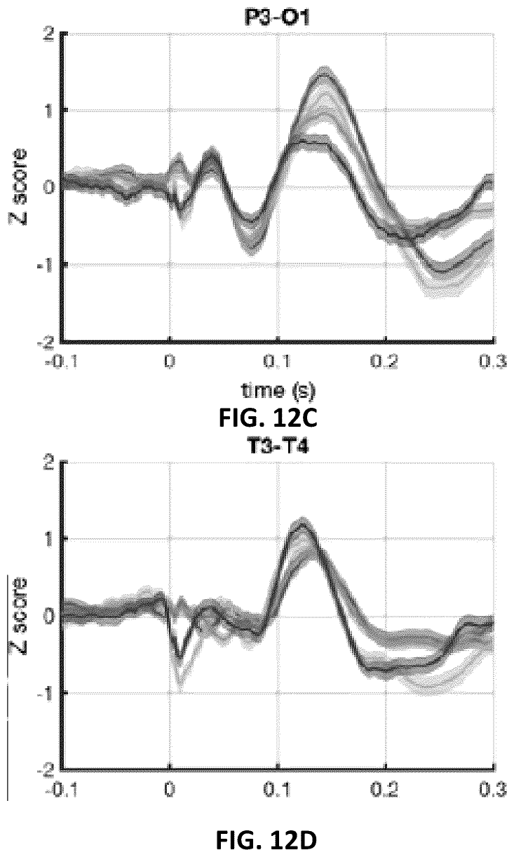

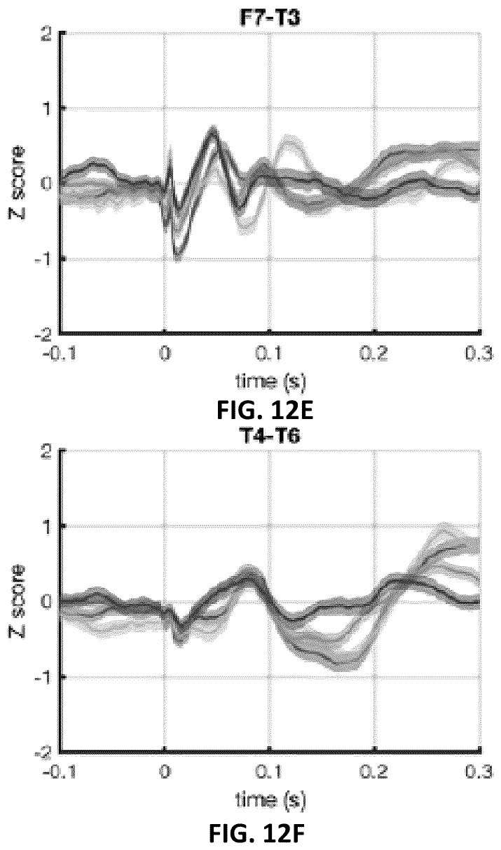

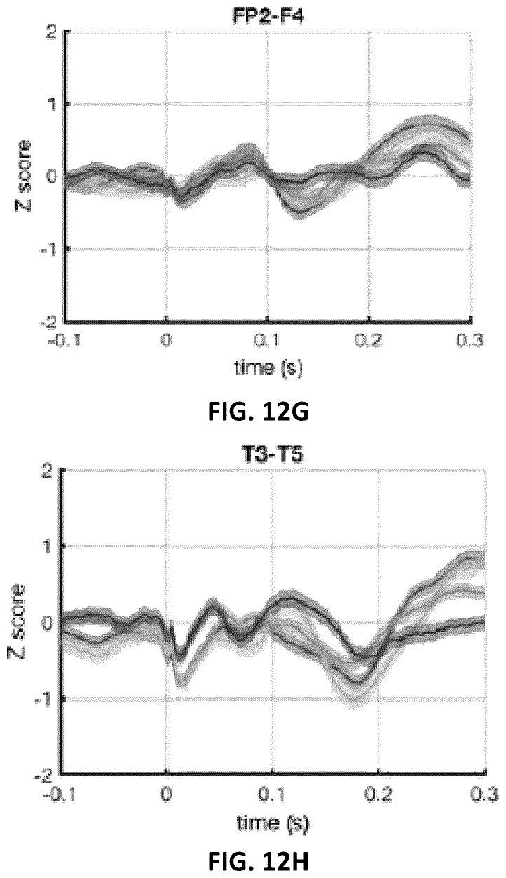

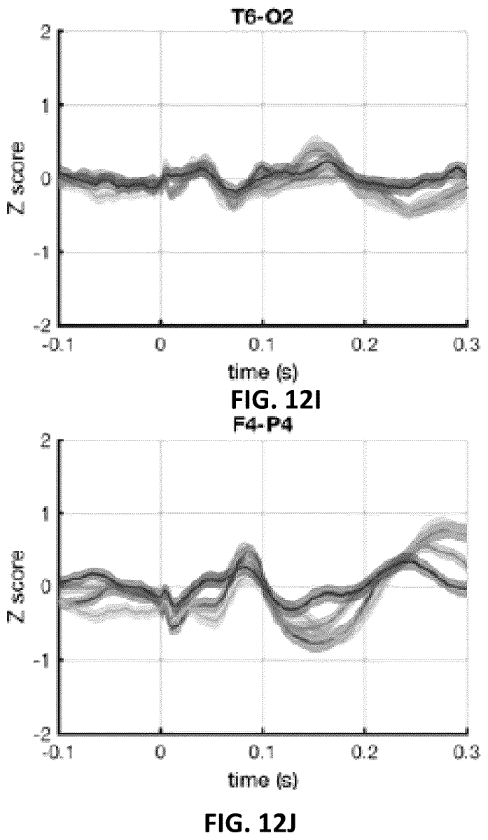

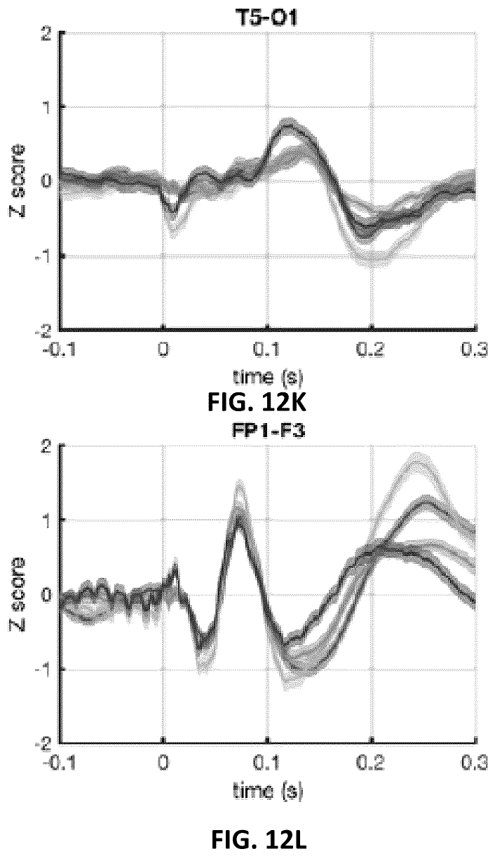

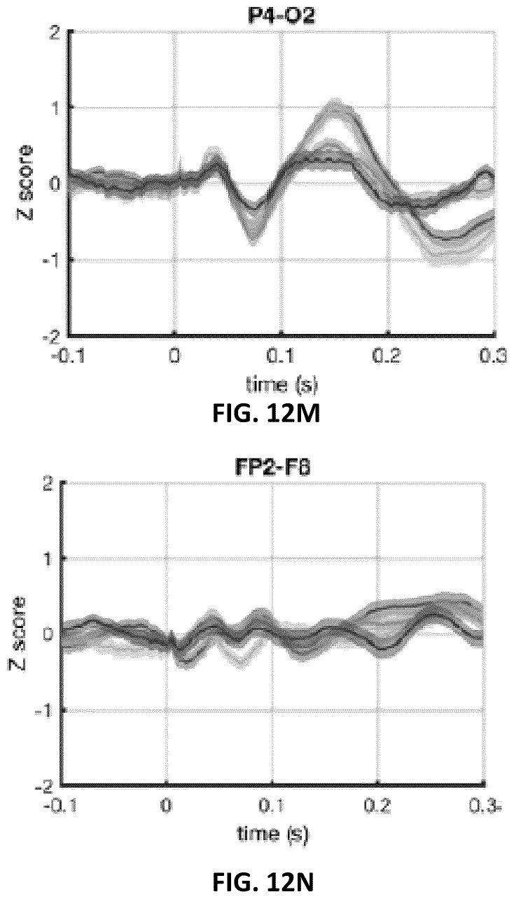

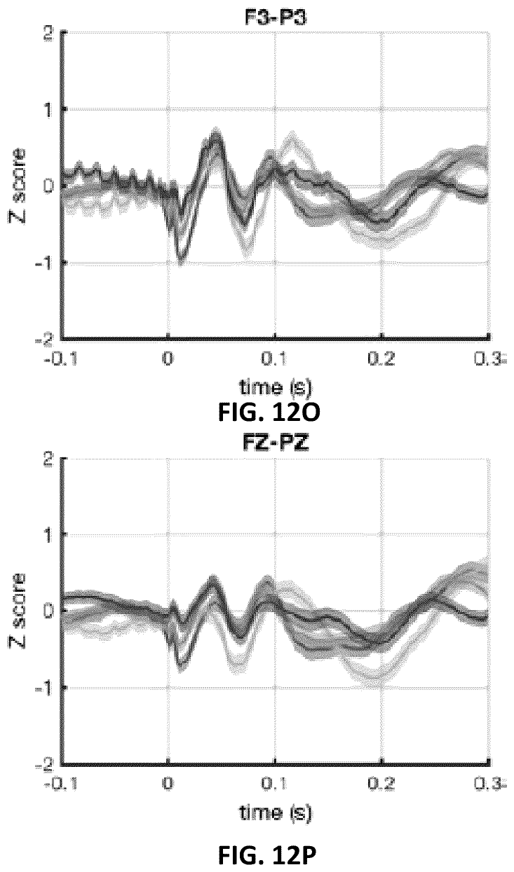

In Example 2, it is reported that stimulating the centro-median thalamic nucleus (FIGS. 12A, 12B, 12C, 12D, 12E, 12F, 12G, 12H, 12I, 12J, 12K, 12L, 12M, 12N, 12O, and 12P) causes widespread network changes, mediating the therapeutic effect. Further, it is reported that the magnitude of EEG responses stemming from electrical stimulation of the CM is a measure of the extent of network activation.

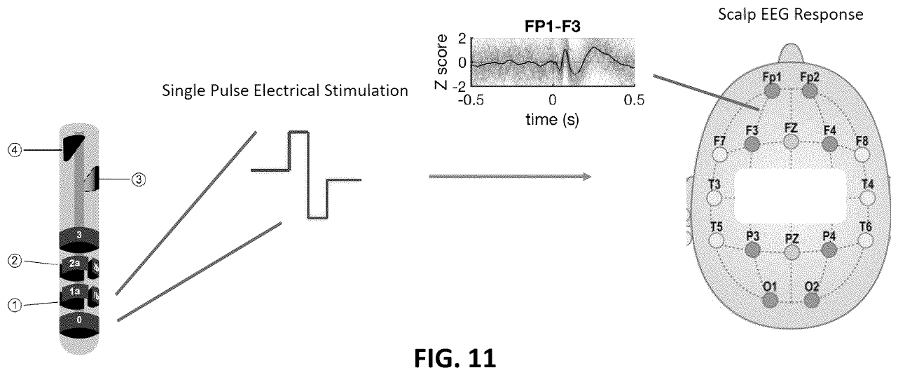

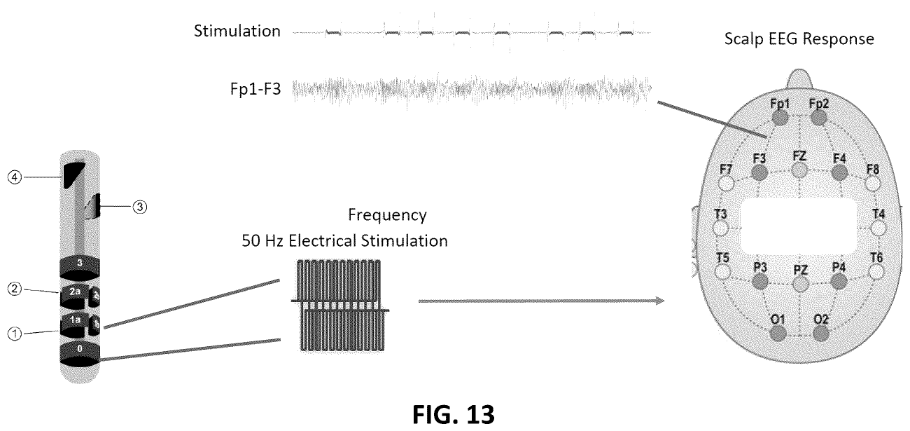

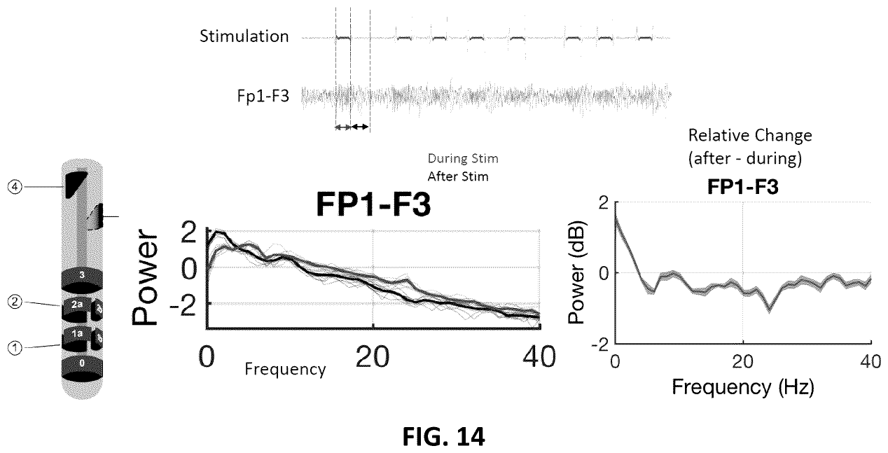

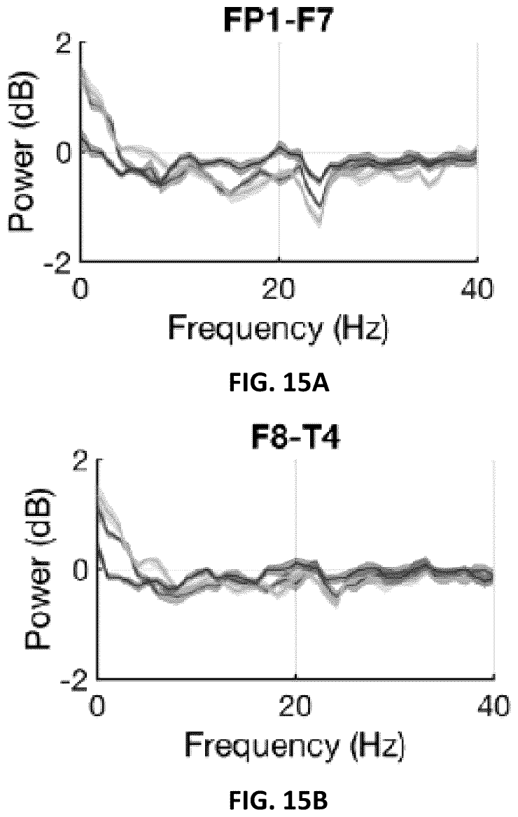

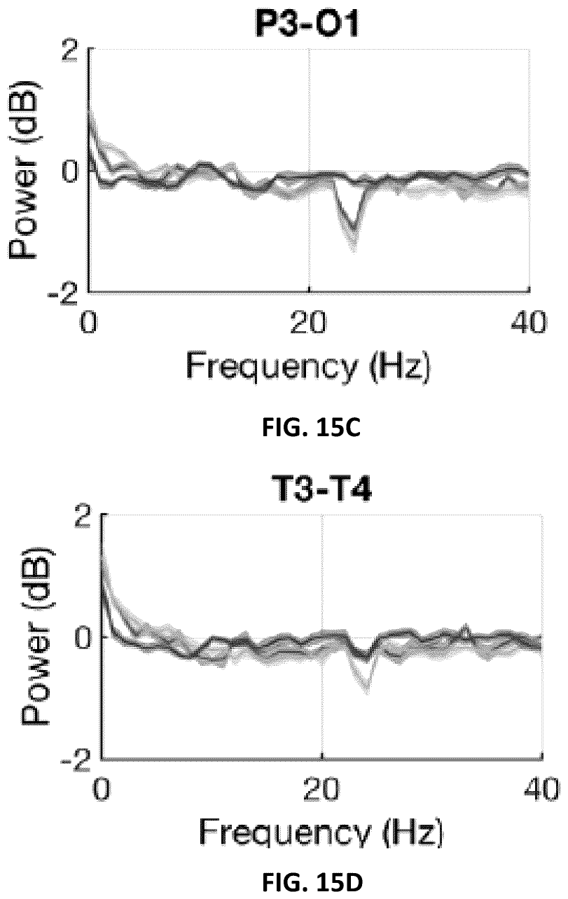

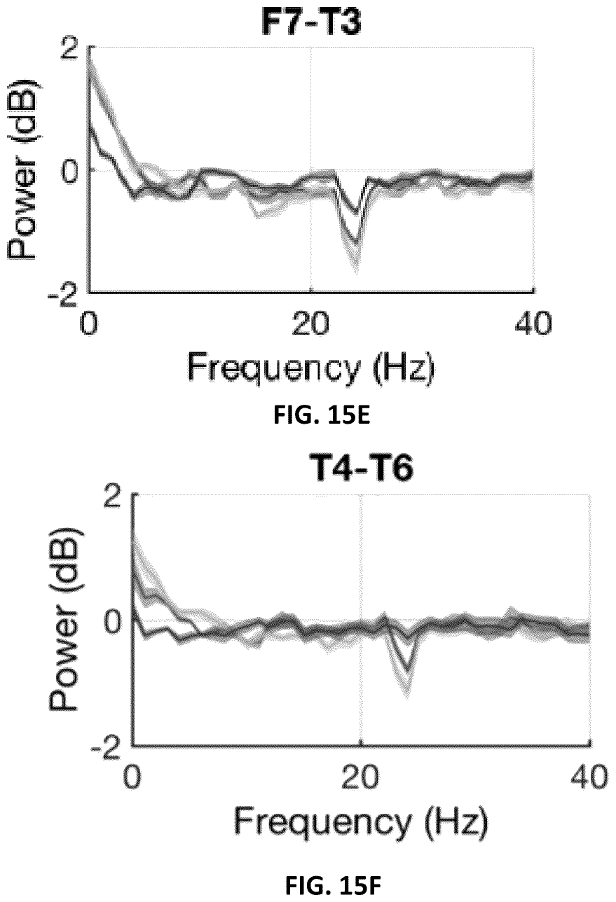

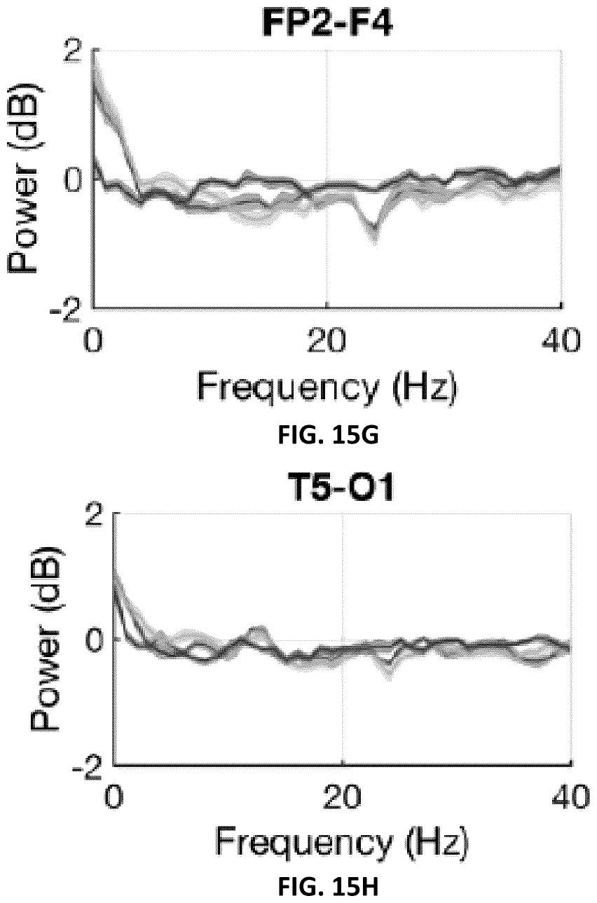

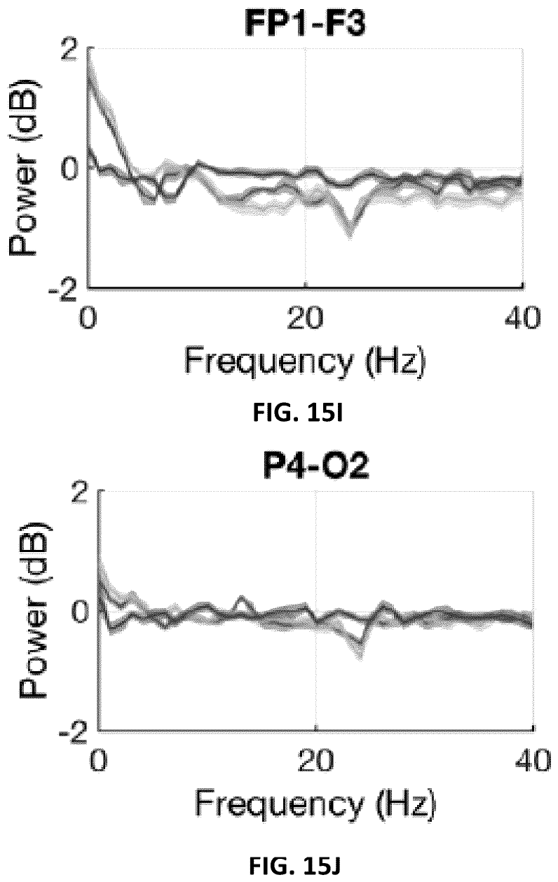

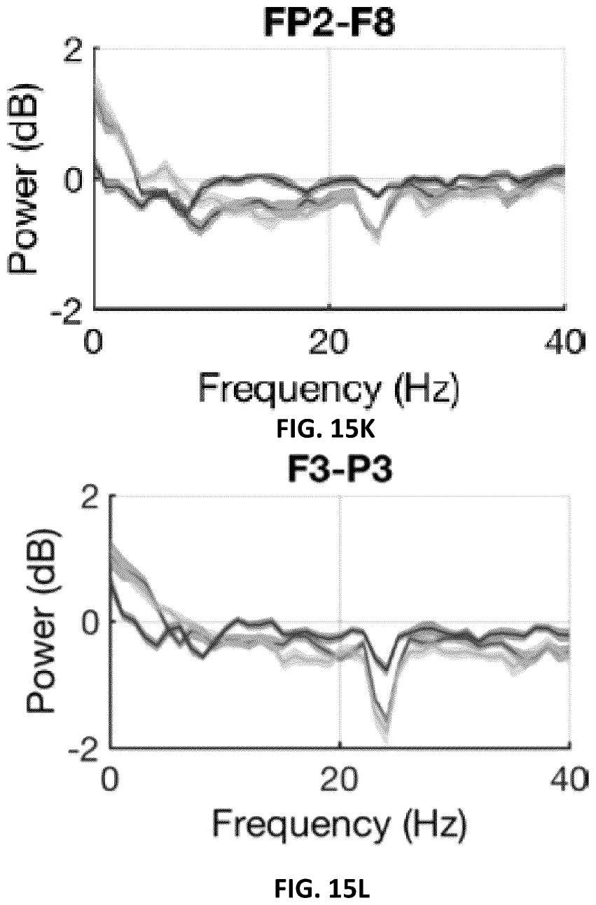

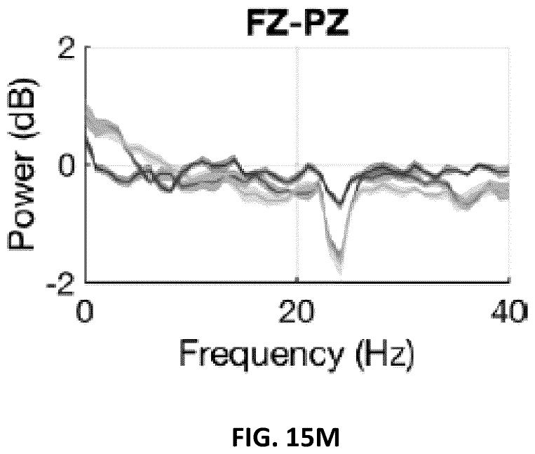

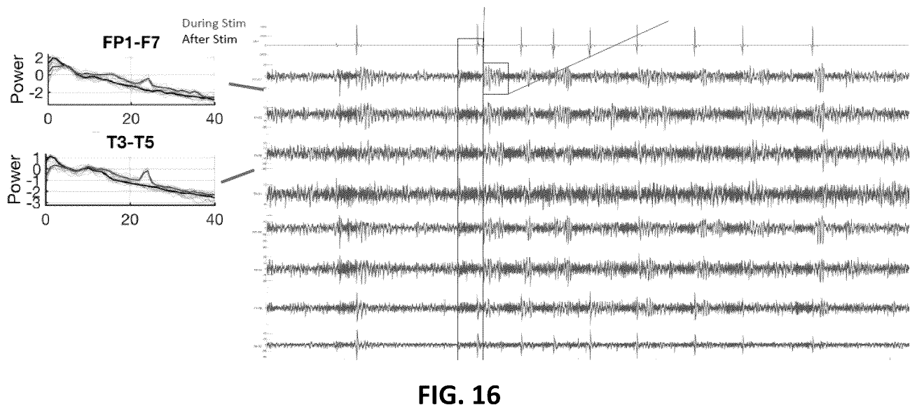

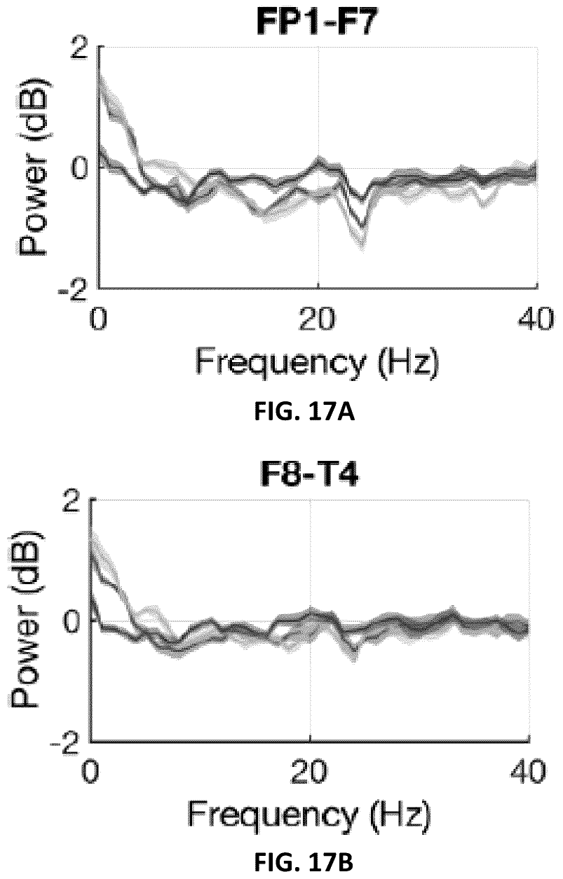

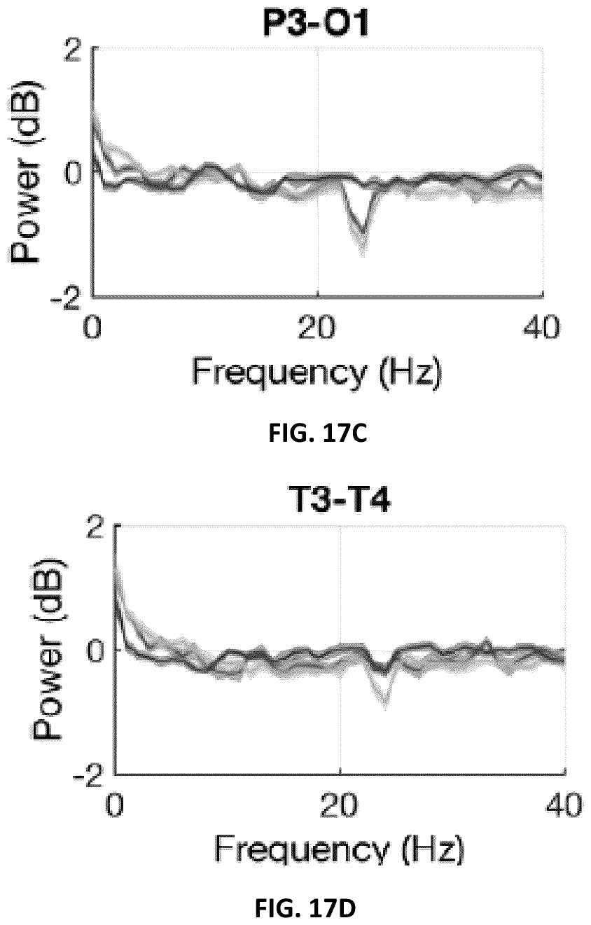

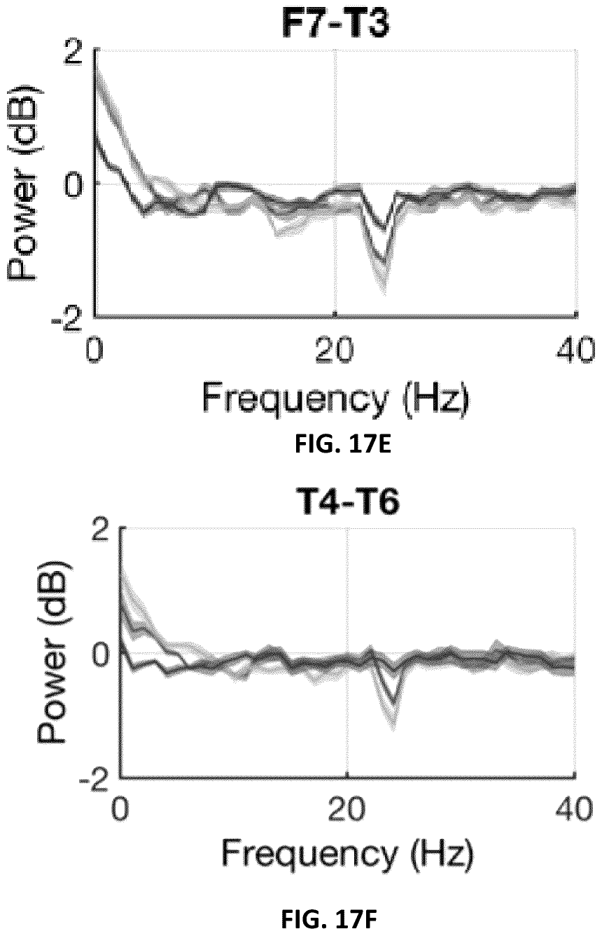

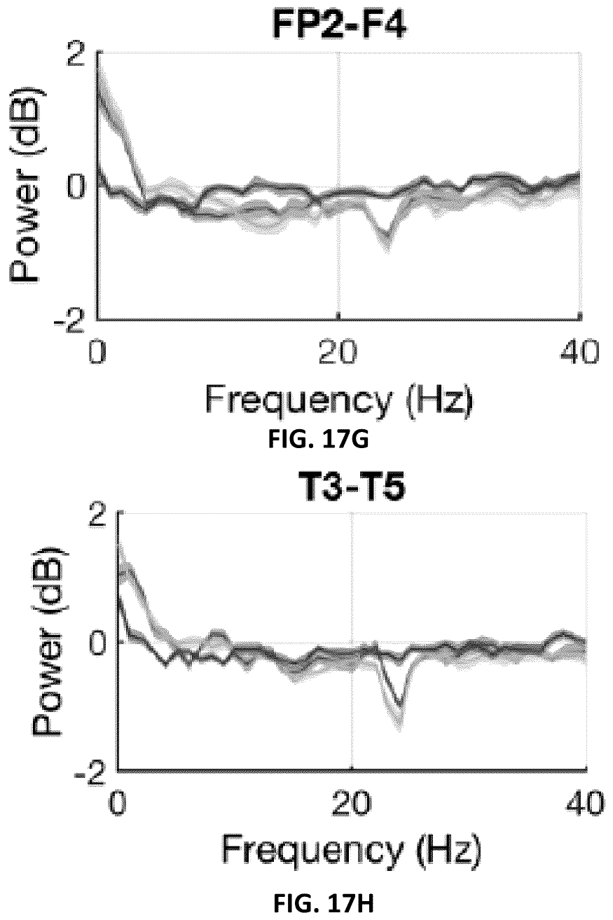

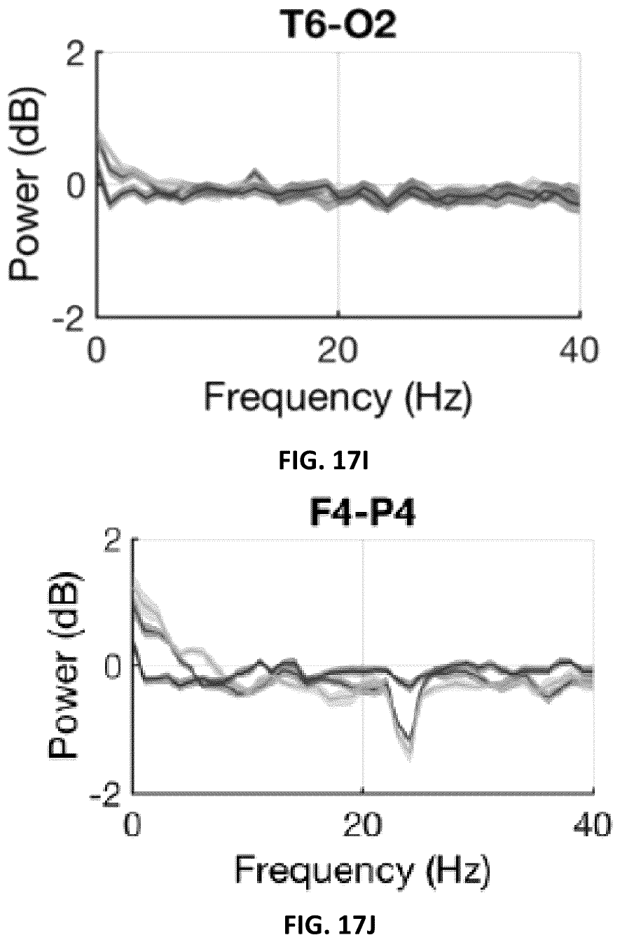

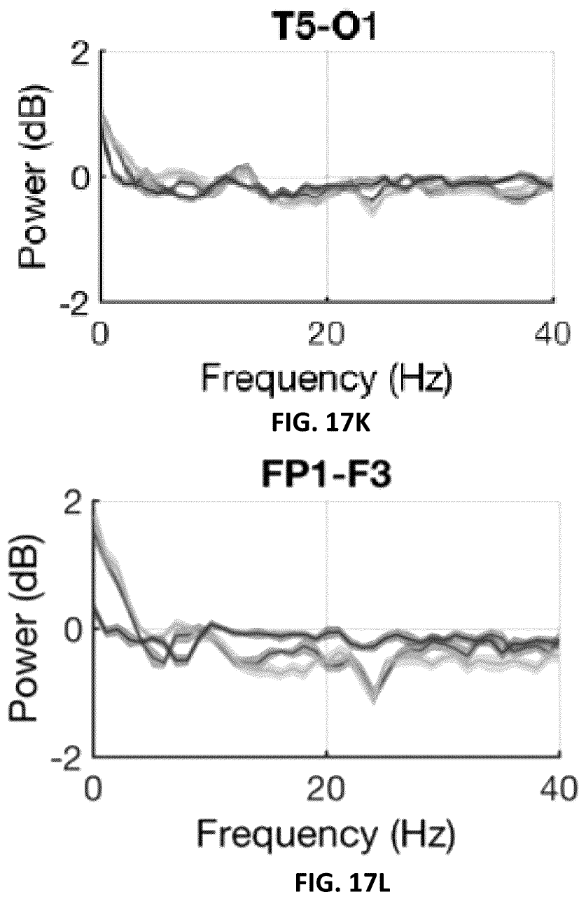

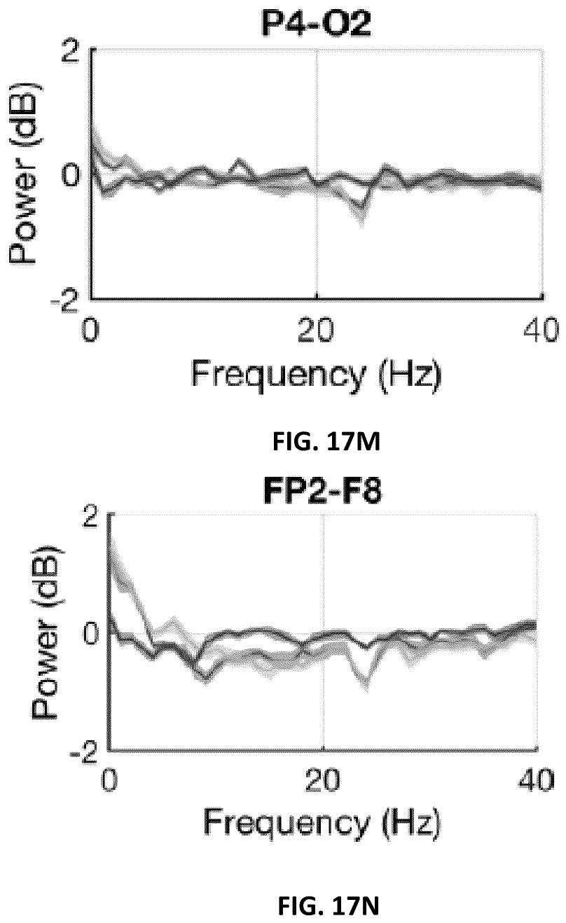

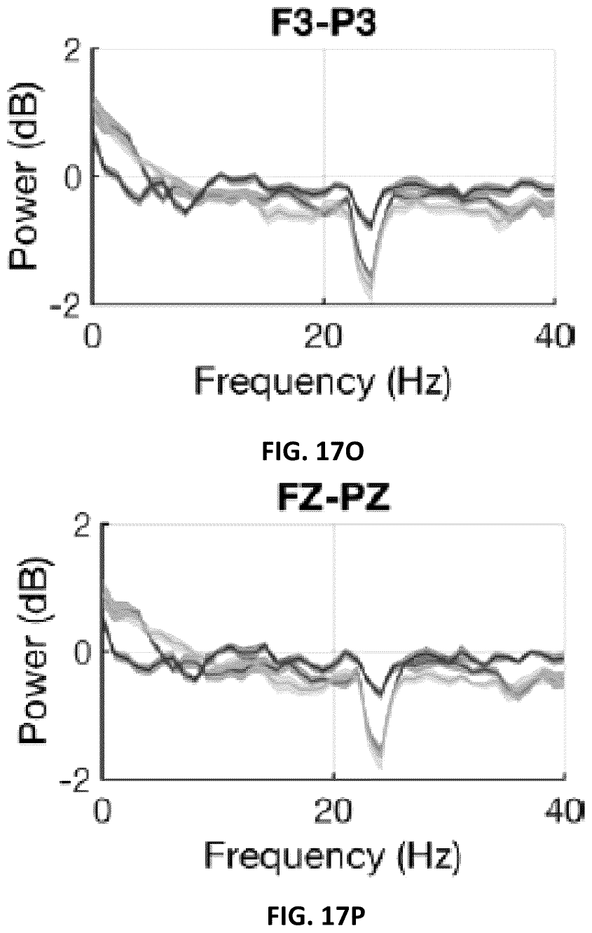

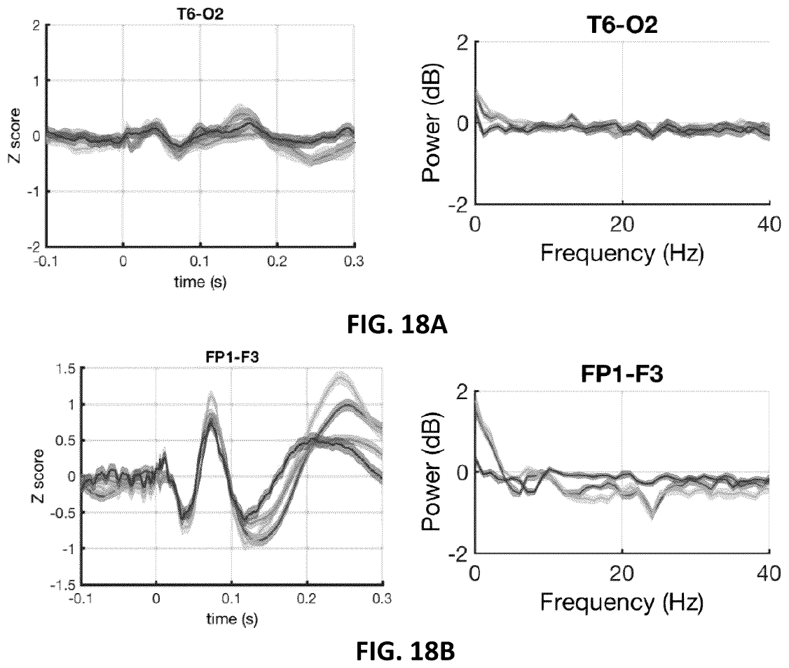

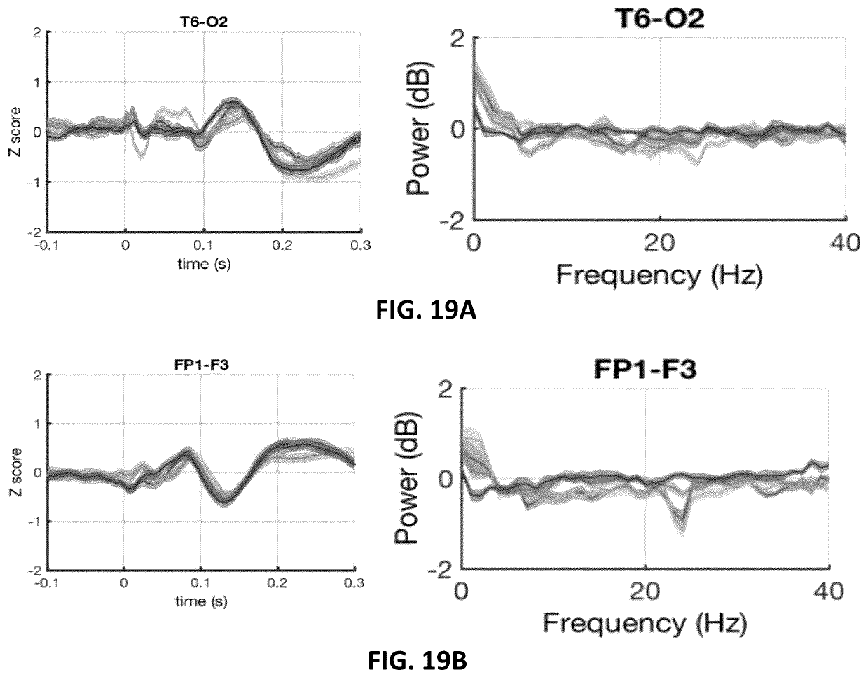

Scalp responses can vary by single-pulse stimulation location (FIGS. 11, 12A, 12B, 12C, 12D, 12E, 12F, 12G, 12H, 12I, 12J, 12K, 12L, 12M, 12N, 12O, 12P, 13, 14, 15A, 15B, 15C, 15D, 15E, 15F, 15G, 15H, 15I, 15J, 15K, 15L, 15M, 16, 17A, 17B, 17C, 17D, 17E, 17F, 17G, 17H, 17I, 17J, 17K, 17L, 17M, 17N, 17O, and 17P). ERP and PSD were compared in both the left (FIGS. 18A and 18B) and the right (FIGS. 19A and 19B) hemispheres.

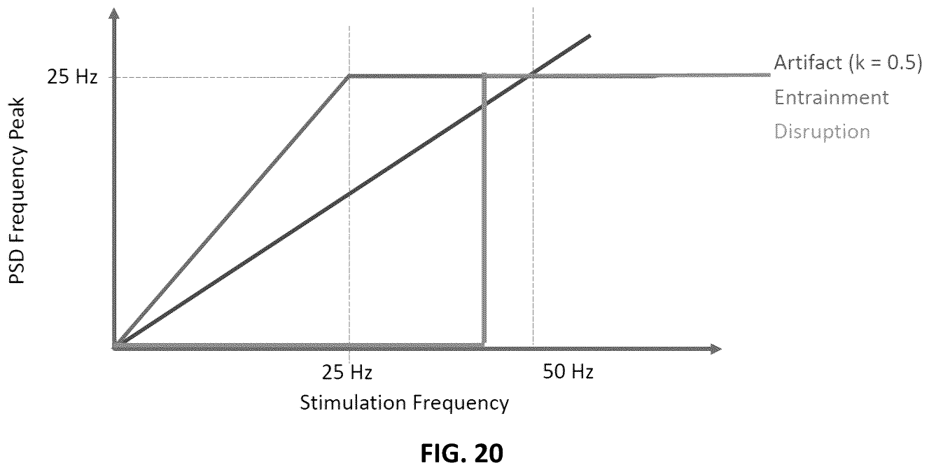

The difference between an artifact, entrainment, or disruption of the thalamo-cortical loop (FIG. 20), as well as a resonance response after stimulation, are discussed.

In conclusion, stimulation location within the centromedial nucleus of the thalamus causes differential effects in scalp recordings, and the more basal contact causes larger responses in the EEG leads (for both 1 Hz and 50 Hz responses).

What is claimed is:

1. An EEG system for facilitating placement of a deep brain stimulation (DBS) device within a brain of a subject to treat a brain disorder, the DBS device configured for implantation within a brain region of the subject and comprising at least two stimulation electrodes configured to deliver a stimulation to the brain region of the subject, wherein the EEG system comprises:
a. an EEG sensing device configured for placement over a scalp of the subject, the EEG sensing device comprising a plurality of EEG electrodes, the EEG sensing device configured to detect a plurality of EEG signals at the scalp of the subject; and
b. a computing device operatively coupled to the DBS device and the EEG sensing device, the computing device comprising at least one processor, wherein the at least one processor is configured to:
i. operate the DBS device to deliver the stimulation to the brain region of the subject;
ii. receive the plurality of EEG signals from the EEG sensing device before and after the stimulation;
iii. transform the plurality of EEG signals received before and after the stimulation into an EEG response biomarker, the EEG response biomarker indicative of a potential extent of neural network interaction accessible by the DBS device; and
iv. display the EEG response marker to an operator of the EEG system.

2. The system of claim 1, wherein the EEG response biomarker comprises a root-mean-square value or a Z-score of an EEG response to the stimulation.

3. The system of claim 2, wherein the EEG response to the stimulation is evaluated within a 500 μs window after the delivery of the stimulation by the DBS device.

4. The system of claim 1, wherein the stimulation comprises a series of single stimulation pulses delivered at a pulse frequency ranging from about 1 Hz to about 190 Hz.

5. The system of claim 4, wherein the single stimulation pulses comprise pulses delivered at 6 mA intensity, 200 μs pulse duration, and 120 seconds stimulation duration.

6. The system of claim 4, wherein the pulse frequency is selected from about 1 Hz, about 25 Hz, and about 50 Hz.

7. The system of claim 1, wherein the EEG sensing device comprises a wearable scalp EEG electrode array.

8. The system of claim 1, wherein the system is configured to operate intraoperatively during implantation of the at least two stimulation electrodes of the DBS device, postoperatively after implantation of the at least two stimulation electrodes of the DBS device; and any combination thereof.

9. The system of claim 1, wherein the at least one processor is further configured to:
a. operate the DBS device to deliver at least two different stimulations to the brain region of the subject, each stimulation delivered by a different pair-wise combination of the at least two electrodes;
b. compare at least two EEG biomarkers, each EEG biomarker corresponding to each pair-wise combination of the at least two electrodes;
c. select a final pair-wise combination of stimulation electrodes for subsequent DBS treatment of the subject, the final pair-wise combination of stimulation electrodes corresponding to the maximum EEG biomarker value; and
d. display to the operator of the system the final pair-wise combination of stimulation electrodes for use in subsequent DBS treatments.

10. A computer-implemented method of positioning and configuring the operation of a DBS device within a brain region of a subject, the method comprising;
a. positioning an EEG sensing device comprising a plurality of EEG electrodes and configured to detect a plurality of EEG signals on a scalp of the subject, and implanting the DBS device comprising at least two stimulation electrodes within the brain region of the subject;
b. obtaining, using the computing device, at least two candidate EEG biomarker values during implantation of the DBS device within the brain region of the subject, wherein the at least two candidate EEG biomarker values are obtained for at least one DBS treatment parameter;
c. comparing, using the computing device, the at least two EEG biomarker values to identify a maximum EEG biomarker value; and
d. configuring the operation of the DBS device to match the at least one treatment parameter of the DBS device associated with the maximum EEG biomarker.

11. The method of claim 10, wherein obtaining at least two candidate EEG biomarker values comprises, for each candidate EEG biomarker value:
a. operating the DBS device to deliver the stimulation to the brain region of the subject;
b. receiving, using the computing device, the plurality of EEG signals from the EEG sensing device before and after the stimulation;
c. transforming, using the computing device the plurality of EEG signals received before and after the stimulation into one candidate EEG response biomarker, the one candidate EEG response biomarker indicative of a potential extent of neural network interaction accessible by the DBS device; and
d. display the one candidate EEG response marker to an operator of the EEG system.

12. The method of claim 10, wherein at least one DBS treatment parameter is selected from:
a. one or more positions of the at least one stimulation electrodes within the brain region of the subject;
b. one or more pair-wise combinations of the at least two stimulation electrodes of the DBS device;
c. one or more combinations of DBS operating parameters; and
d. any combination thereof.

13. The method of claim 12, wherein the DBS operating parameters are selected from a pulse frequency, a pulse duration, a pulse amplitude, a treatment duration, and any combination thereof.

14. A computer-implemented method of monitoring an efficacy of a DBS treatment of a brain disorder of a subject using an EEG system comprising a computing device operatively coupled to an EEG sensing device configured to detect a plurality of EEG signals on a scalp of the subject, wherein the method comprises:
a. administering a first DBS treatment and obtaining a first EEG biomarker using the EEG system;
b. administering a second DBS treatment and obtaining a second EEG biomarker using the EEG system;
c. obtaining, using the computing device, a change in the second EEG biomarker value relative to the first EEG biomarker value; and
d. evaluating, using the computing device, the efficacy of the DBS treatment based on a predetermined correlation of the efficacy and the change in the second EEG biomarker value relative to the first EEG biomarker value.

15. The method of claim 14, wherein the brain region comprises a centromedian thalamic nucleus region.

16. The method of claim 14, wherein the brain disorder comprises one of Parkinson's disease and epilepsy.
